(19) United States
(12) Patent Application Publication
(10) Pub. No.: US 2025/0288583 A1
(43) Pub. Date: Sep. 18, 2025

(54) Treatment of Mucus Hypersecretion

(71) Applicant: Edward Tak Wei, Berkeley, CA (US)

(72) Inventor: Edward Tak Wei, Berkeley, CA (US)

(21) Appl. No.: 18/445,901

(22) Filed: Mar. 14, 2024

(51) Int. Cl. A61K 31/505; A61K 9/00; A61K 31/167; A61K 31/223; A61K 31/4402; A61P 11/02

(52) U.S. Cl. CPC A61K 31/505; A61K 9/0043; A61K 9/0078; A61K 31/167; A61K 31/223; A61K 31/4402; A61P 11/02

(57) ABSTRACT

Patients suffering from rhinitis and rhinosinusitis often experience symptoms such as nasal discharge, congestion, sniffles, and excess phlegm secretion. These symptoms can cause a feeling of impaired airflow, loss of patency, and uncomfortable breathing. In this context, a treatment method is proposed to alleviate these signs and symptoms. The drug target is the TRPM8 receptor on nerve fibers of the ostiomeatal complex. The active ingredient, a TRPM8 agonist, is delivered using an electronically controlled nebulizer that produces a vigorous air plume of defined particle sizes, which prevents entry into the lungs. The quantity of phlegm expelled from the nasal passage is used as an index of anti-inflammatory effectiveness. The optimized drug candidates for delivery and therapy are from a group of p-menthanecarboxamides. Preferred embodiments of these include the p-menthane amino acid esters: GlyOnPr, GlyOiPr, D-Ala-OnPr, and D-Ala-OiPr, delivered as a liquid suspension or solution at 0.1 to 1% (1 to 10 mg/mL), 0.5 to 0.8 mL per nostril over 3 to 5 seconds, using a nebulizer. This regimen, administered three times per day for three to five days, will help the patient manage and control their nasal congestion and phlegm secretion, and sleep better at night. The preferred embodiments delivered into the nasal cavity create a sense of cool breathing, refreshing coolness, and a feeling of negative heat flux, like an air conditioner in the nose. The secretion of phlegm is clearly decreased, as measured with a top-loading balance. This pharmacological treatment for patients with nasal inflammation has the potential to alter the course of rhino-disorders permanently.

### CROSS-REFERENCE TO RELATED APPLICATIONS

This application is a continuation-in-part of Ser. No. 18/445,946 filed Jan. 12, 2024.

### BACKGROUND OF THE INVENTION

### Field of Invention

One aspect of the present invention is a method of treatment of mucus hypersecretion in the nasal cavity in a subject in need of such treatment. A typical example of nasal mucus hypersecretion is the excess secretions produced by the common cold virus. This condition is called viral acute rhinosinusitis. Reducing mucus hypersecretion in viral rhinosinusitis relieves nasal stuffiness and allows more comfortable breathing.

### Description of the Related Art

The nasal cavity is a conduit for air into and out of the lungs. The surface area of the nasal cavity is about 150 to 200 cm² for adults (about the size of one surface of the hand). This cavity is lined with a specialized mucosa that warms and humidifies the inhaled air. Mucosal secretions come from serous, mucinous, and sero-mucinous exocrine glands. Serous fluids are transparent and watery and contain electrolytes and proteins, such as amylase and lysozyme. The mucus is thick, gel-like, viscous, and contains mucins (large glycoproteins). The mucus is ˜95% water, but when dried, it forms “snot” and crusts in the nasal cavity. Mucus can be a nutrient source for infections and, together with inflammatory exudates, form “phlegm.” For a healthy and clean nasal cavity, a subject must expel excess phlegm by blowing or swallowing. Otherwise, the phlegm obstructs and, when dessicated, impacts the epithelia and becomes a source of pathophysiology.

The paranasal sinuses are air-filled cavities within the bones of the face and, like the nasal cavity, lined with mucosa and exocrine glands. The sinuses connect to the nasal cavity via apertures called ostia. The ostiomeatal complex (OMC) is an anatomical region in the nasal passages and sinuses (FIG. 1,2) consisting of a series of small openings (ostia) that connect the frontal, maxillary, and anterior ethmoid sinuses to the middle meatus of the nose. Also, part of the OMC is the sphenoidal sinus and posterior ethmoidal cells that drain into the superior meatus. Mucus hypersecreted into the nasal cavity and sinuses will accumulate and impede breathing unless removed. It has been recognized for some time that excess mucus is not healthy for the airways, but effective methods for reducing mucus in airways are limited.

### BRIEF SUMMARY OF THE INVENTION

One aspect of the present invention is a method of treatment of mucus hypersecretion in the nasal cavity, to reduce phlegm secretion, in a subject in need of treatment thereof. The embodiment is a p-menthanecarboxamide TRPM8 agonist, formulated in a liquid suspension or as a solution, and topically administered onto the ostiomeatal complex of the subject's nasal cavity with an electronically controlled nebulizer or a manually operated sprayer. Objective evidence of reduced mucous secretions is obtained by weighing the nasal blowouts of the treated subject. The discovery was that the topical medication inhibited nasal secretion of phlegm. Beneficial effects were observed within 15 min of delivery of the preferred embodiment and lasted for about six hours after a single delivery: Multiple doses appear to confer permanent benefits for subjects with chronic mucus hypersecretion.

When secreted by the mucous gland, mucus is a transparent white protein, but when mixed with other secretions and inflammatory exudates, it becomes nasal phlegm. This phlegm is the principal pathophysiological element in rhinitis. Nasal phlegm is the yellowish-green slimy substance copiously secreted by the glands of the nasal mucosa when inflamed. The principal element of phlegm is a mucopolysaccharide called MUC5B, which absorbs water from the environment and swells to form a gel. When the gel dries, it becomes a crusty snot and can be as hard as shoe leather. Removal of excess phlegm is necessary for a healthy and patent nasal passage. Otherwise, it will coat and obstruct airflow, impede thermal exchange in the nasal cavity, and interfere with mucociliary clearance. The opaque materials in an X-ray that show blocking of the nasal passages and sinuses and defining the state of severe chronic rhinitis is desiccated phlegm.

The anatomical target for sustained relief of nasal stuffiness and exudate secretions in the practice of the present invention is the TRPM8 receptors on nerve fibers of the ostiomeatal complex (OMC) (FIG. 1, 2). The OMC is a central region in the nasal passages and sinuses. It consists of a series of small openings (ostia) that connect the frontal, maxillary, and anterior ethmoid sinuses to the middle meatus of the nose. The OMC is posterior to the nasal valve (the narrowest area of the nasal cavity with an area of about 0.7 cm², about 1.3 cm behind the nares). This surface is not easily accessible with conventional sprays or nose drops. An electronically controlled nebulizer for drug delivery is the preferred method of topical administration of the preferred embodiments. Successful treatment of patients with mucus hypersecretion means relieving symptoms of nasal stuffiness. Surprisingly, this treatment had a cumulative beneficial effect, such that after a 3 to 5-day course of drug treatment, symptoms of rhinosinusitis were well controlled.

A nebulizer and a sprayer disperse liquids into droplets, but they operate differently. A nebulizer is conventionally used to deliver medication as a mist inhaled into the lungs. Such formulations and methods are common for asthma, chronic obstructive pulmonary disease, or other lower respiratory disorders. Nebulized aerosols enter via the oral cavity. A sprayer is a general term for any device dispensing aerosols over a surface. Hand-held sprayers are often used medically for delivering liquids into the nasal cavity, with the tip inserted into the nostril. The sprayer uses manual air pressure to force the liquid out of the nozzle, breaking it into airborne droplets. The aerosol disperses over the target area. Usually, the particle size distribution, dispersion pressure, and volume of a hand-held sprayer are not exact because of user differences. Hand-held sprayers are used to deliver intranasal steroids, antihistamines, and decongestants for the treatment of rhinitis. These drugs are effective for allergic rhinitis but not viral rhinitis. The steroids have some efficacy for chronic rhinosinusitis.

Flickinger (U.S. Pat. Nos. 5,906,198 and 8,892,544) described a electronic nebulizer optimized for selectively delivering particles into the nasal and paranasal cavities. This system utilizes an electronically controlled compressor to generate large liquid particles. Over 99.9% of the particles are >10μ. These particles get trapped in nasal and sinus cavities and do not enter the lungs. This system is available as NasoNeb® Nasal Nebulizers. The nebulizer used here is Model 7070NasoNeb® Sinus Therapy System. The use of this device helped enable the discovery of this application. The nasal and sinus cavities capture virtually all particles delivered via the NasoNeb Nebulizer (FIG. 3). Under standardized compression, the plume going past the nasal valve is retained in the middle meatus and delivered to the OMC with maximum effect (FIG. 3). The areas reached by the nebulized large particles include the frontal recess/sinus, spheno-ethmoid recess, ethmoid cavity, sphenoid and maxillary sinuses, turbinates, the middle meatus, and olfactory cleft. Conventional manual sprayers and Metered Dose Inhalers (MDIs) produce smaller particles that mainly reach the anterior third of the inferior and middle turbinates. These devices do not have a positive pressure generator and lose momentum after exiting the nozzle. The pump creates positive pressure and an air column for the nebulizer to propel particles past the nasal valve and deeper into the nasal cavity. The particle size distribution with the nebulizer is such that particles are less likely to reach the pharynx and to cause adverse effects such as laryngeal irritation or voice alterations.

The precise electronic control of the compressor in the nebulizer allows accurate calculation of the administered dose. Experimentally, the nebulizer delivers 0.35 to 0.75 mL into the nasal cavity in a 3 to 5-second activation. This control of the rate and volume of liquid delivery permits quantitative comparisons of the specificity and selectivity of the preferred embodiments. In the future, the parameters of the nebulizer pump may be adjusted for various delivery times, e.g., 5 to 10 sec, but preserving the desired particle size distribution. A lightweight hand-held device weighing ˜300 g, with a reservoir canister of 5 mL and a battery power source, may be optimal for individual use.

A top-loading balance, sensitive to 1 mg, was used to measure phlegm secretions from the nose. A pre-weighed tissue paper was tared to zero, and after blowing into the tissue, the amount of exudate was recorded from the balance display. This measurement is an objective sign of nasal inflammation. Microbalances, sensitive to 1 mg, are now available online for less than $20. Conventional laboratory balances with the same capabilities are listed for $2000+ each but should now be obsolete. The convenience of an inexpensive method for quantifying phlegm output as an indicator of nasal pathology is part of the inventive steps.

In the past 20+ years, scientists have realized that cooling is a molecular event conveyed by dedicated nerve fibers containing TRPM8 (an integral membrane protein receptor). These nerve fibers fire when tissue temperatures drop below 25° C., stimulated by the negative heat flux or chemical agents. The cooling effect is modality-specific, and its anatomic circuitry has its own set of cables. TRPM8-mediated cooling is the physiological basis for “cool esthesia.” TRPM8 agonists replicate a negative heat flux sensation from the nasal membranes. Surprisingly, the studies here show that when these molecules are rinsed onto the nasal cavity surfaces, excess phlegm production dramatically declines. The experiments for finding an effective molecule and the appropriate delivery parameters are described here.

In summary, the drug target is the TRPM8 receptor on nerve fibers of the OMC (FIG. 1,2): The active ingredient is a TRPM8 agonist. FIG. 1 shows a photomicrograph of the TRPM8 target in a nerve fiber on the mouse nasal mucosa. The method of drug delivery is an electronically controlled nebulizer (FIG. 3). The choice of the best TRPM8 agonist is from an arsenal of molecules known to the art or synthesized de novo by the applicant. The quantification of response is the output of nasal exudates measured with a top-loading microbalance. Examples of optimized candidates for delivery and therapy are presented.

A nebulizer with an electronically controlled compressor was used to deliver the cooling agent (liquid suspension) to the ostiomeatal complex (OMC). Examples of preferred embodiments for the practice of this invention are the p-menthane amino acid esters GlyOnPr, GlyOiPr, D-Ala-OnPr, and D-Ala-OiPr (Table 10) delivered to the OMC, and other p-menthanecarboxamides that target TRPM8. A typical dosage regimen was a 0.2 to 1.0% wt/vol solution (2 to 10 mg/mL) at a volume of 0.2 to 0.5 mL/nostril over a 3 to 5 sec delivery period with a nebulizer. A dosage schedule of two to three times per day for three to five days lowers phlegm output, relieves symptoms and allows the subject to manage and control mucus hypersecretion and sleep well at night.

### BRIEF DESCRIPTION OF SEVERAL VIEW OF THE DRAWINGS

FIG. 1. Photomicrograph of the TRPM8 receptor on mouse nasal mucosa nerve fiber [Wei. Medical Hypotheses 142 (2020) 109747]. The histology methods are described in Yang et al., 2017. The TRPM8 fiber was identified by green fluorescent protein methods in transgenic mice (Trpm^{8+/EGFP}). The TRPM8 receptor is the drug target.

FIG. 2. The ostiomeatal complex (OMC) is the anatomic site for intended drug delivery. An electronic nebulizer is used to spray droplets over the OMC at a controlled rate with positive pressure. The lateral walls of the OMC contain the ostia of the sinuses, three in the middle meatus and two in the superior meatus. Delivery of the TRPM8 agonist is intended to relieve stuffiness and facilitate drainage of the sinuses via the ostia. The anatomic complexity of the OMC is apparent in this illustration from Gray's Anatomy. A manual sprayer will deliver droplets to the anterior ⅓ of the cavity, primarily in the antrum. Instilled liquid drops will mainly flow along the inferior meatus and exit in the choanae. A positive pressure rinse is optimal for delivery to the OMC.

FIG. 3. The delivery unit for the preferred embodiments. The left panel is the NasoNeb® nebulizer (used in the present studies), wherein >99% of the particles generated from the canister are >10μ in diameter and thus do not enter the lungs. The squeeze bottle contains the test solution to fill the canister. The liquid plume from the electronically controlled nebulizer is more powerful than conventional manual sprayers or irrigators. Rinsing the nasal cavity with a liquid suspension of the preferred embodiments over three to five seconds per nostril is sufficient for efficacious treatment.

FIG. 4. Agonist activities of TRPM8 analogs. A standard method for receptor activity is to measure the median effective dose (EC₅₀) as an indicator of potency. The relative fluorescence (RF) unit on the ordinate represents calcium entry into transfected cells. The data points are for: D-Ala-OEt ▴, D-Ala-OiPr ●, GlyOiPr ▪, D-Ala-nBu⋄L-menthol ◯, and L-Ala-OEt ▾, respectively. The structures of these compounds are in Table 4. GlyOiPr is 7.6× more potent than l-menthol on TRPM8.

FIG. 5. Specificity of GlyOiPr for TRPM8 but not TRPV1 or TRPA1. Cells transfected with the TRPV1 (▴, Δ) or TRPA1 (●, ◯), respond to the positive control substances capsaicin and mustard oil (filled symbols ▴,●) but do not respond to the preferred embodiments (Δ, ∘). GlyOiPr ▪ and l-menthol ▾ are active on TRPM8 transfected cells.

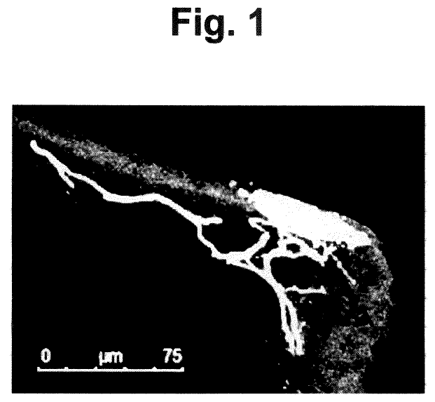

FIG. 1. Comparison of cooling duration of various p-menthanecarboxamide analogs on philtrum skin versus ocular margins. The philtrum skin is keratinized, but the ocular margins are not. The test substances numbered 1 to 11 are: D-Ala-OiPr, Gly-OiPr, D-Ala-OEt, D-Ala-OMe, D-Ala-GlynPr, GlynPr, D-Ala-OnBu, GlynBu, GlyOEt, L-Ala-OEt, and N-Me-D-Ala-OEt, respectively. The structures and data are shown in Table 4, 6 and 7. Two outliers (1 and 2) were identified as preferred embodiments because they preferentially cool the ocular margins. The nasal cavity surfaces like the ocular margins are not keratinized.

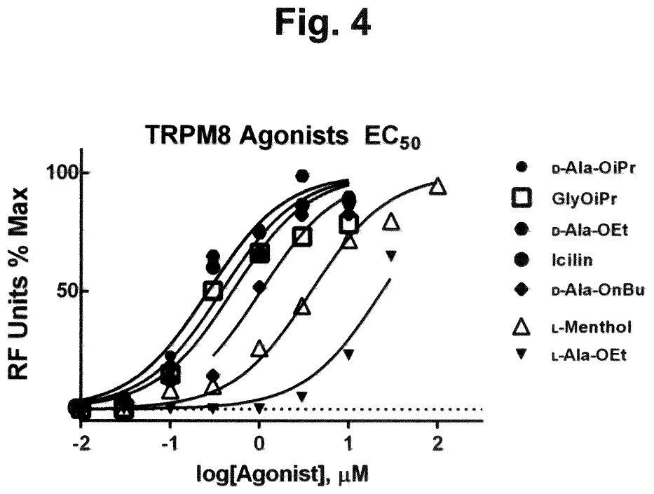

FIG. 7. Symptoms of the common cold (from pg. 55, FIG. 4.1.3 of Fokkens et al., 2020). The chart illustrates the time course of changes after an episode of viral-rhinosinusitis. Increased nasal discharge is the most common sign of infection.

FIG. 8. Measurement of secretions and exudates from the nasal cavity. Subjects were instructed to blow into a pre-weighed paper tissue and record the weight of the tissue after blowing. The tissue was inspected and marked as clear, with streaks of exudates, or dense gel. Surprisingly, the nasal blowouts fell into three distinct categories of weights that were easy to recognize. The relative unit weights (mean±s.e.m.) are: clear, 0.06±0.01 g, streaks 0.20±0.01 g, and dense gel 0.47±0.03 g. The weight of the blowouts provides an objective index of nasal cavity inflammation.

FIG. 9. Inhibition of nasal secretions in a subject with rhinitis by topical administration of the preferred embodiments D-Ala-OiPr and GlyOiPr with a Nasoneb® apparatus. Rinsing of GlyOiPr and D-Ala-OiPr (dissolved 3 mg/ml and 2 mg/mL, respectively, in 1% ethanol and 2% polysorbate 80) was for 5 sec at t=0, 2, and 5 hr. Secretions were recorded for 10-15 hr per day. The 10-hr secretion (avg.±s.e.m) was vehicle 3.53±0.44 g, D-Ala-OiPr 1.70±0.27 g, GlyOiPr 1.32±0.32 g, with n=6, 5, 5 observations per group, respectively. The 10 hr unit exudation rate for D-Ala-OiPr and GlyOiPr were significantly lower (P<0.001) than the vehicle when analyzed by the Kruskal-Wallis test for non-parametric data.

FIG. 10. Weights of individual blowouts of nasal secretions in a subject with rhinitis after topical administration of D-Ala-OiPr and GlyOiPr with a Nasoneb® apparatus. The vehicle or test substances were rinsed into the nasal cavity (0 mg/mL, 2 mg/mL and 3 mg/mL, respectively, for 5 sec at t=0, 2, and 5 hr). The data points are for individual “blowouts” from the nose, measured with tissue paper and balance. The data are plotted on a semi-log scale, so zero values are not shown. The average #s.e.m. for blowouts were: vehicle 0.40±0.03 g, D-Ala-OiPr 0.19±0.02 g, GlyOiPr 0.19±0.02 g, with n=70, 44, 42 observations per group, respectively. The blowout unit for D-Ala-OiPr and GlyOiPr were significantly lower (P≤0.0001) than the vehicle when analyzed by the Kruskal-Wallis test for non-parametric data.

FIG. 11. Airway mucus secretion and hypersecretion (from Rogers, 2007). Mucus forms a layer over the epithelium. Mucins secreted by goblet cells and submucosal glands confer viscoelasticity on the mucus, which facilitates mucociliary clearance of inhaled particles and irritants. Mucus hydration is regulated by salt (and, hence, water) flux across the epithelium. The glands also secrete water. Plasma proteins exuded from the tracheobronchial microvasculature bathe the submucosa and contribute to the formation of mucus. The above processes are under the control of nerves and regulatory mediators. Note that “congestion” can occur after plasma exudations and be independent of mucus hypersecretion.

### DETAILED DESCRIPTION OF THE INVENTION

### Nasal Pathophysiology: Mucus Hypersecretion, Rhinitis, Congestion, Stuffiness

Patients with rhinitis and rhinosinusitis complain of “stuffiness,” impaired airflow, loss of patency, and uncomfortable breathing. For example, in a survey of 2500 adults with allergic rhinitis, the question, “When you have nasal allergy attacks, which symptom is most bothersome?” The answers were: stuffed-up nose (78%), runny nose (62%), post-nasal drip (61%), red itching eyes (53%), headache (51%), repeated sneezing (51%), watering eyes (51%), itching (46%), facial pain (43%), and ear pain (30%). The sense of blocked breathing is conspicuous in acute viral rhinosinusitis (e.g., the common cold). When one tries to sleep with the common cold, breathing is difficult and mouth breathing makes things worse. In chronic rhinosinusitis, the primary complaints are nasal blockage and mucopurulent rhinorrhea/post-nasal drip.

The term “congestion” is often used interchangeably with stuffiness. However, congestion more precisely refers to evidence of physical blockage or obstruction of the nasal passage due to swelling or excess fluids. Clinicians seldom achieve direct measurements of fluid congestion. Stuffiness can occur without congestion because it is the activity of nerves in the mucosa that provokes stuffiness. Stuffiness is the bane that affects the patient's quality of life. Patients recognize and focus on this complaint. The nasal dynamics leading to the subjective sense of stuffiness and congestion are poorly understood. Congestion is estimated from nasal cavity volume (acoustic rhinometry), nasal airflow resistance (peak inspiratory flow), nasal cavity imaging (CT scans), and infrared thermography. However, none of these parameters showed a tight correlation to stuffiness. Loss of nasal patency can occur without changes in these parameters because nerve signals determine stuffiness. In summary, nasal stuffiness is a subjective perception of impaired breathing, whereas nasal congestion is a physical measurement of impaired flow caused by inflammation. While stuffiness and congestion often occur together, they may result from independent mechanisms.

The subjective sensation of nasal patency was clarified by Shen et al. (Facial Plastic Surgery 33:372-77, 2017). They showed, using sophisticated methods of computerized fluid dynamics, CT scans, and thermography, that heat flux over the anterior one-third of the nasal mucosa was a key mechanism for open breathing. Heat flux refers to the rate of heat (energy) transfer per unit area and time. If the heat flux was negative (cooling), the subject felt more open breathing, like being in front of an air conditioner. The opposite is when wearing a facemask in a hot environment; heat flow is against a higher gradient, and heat accumulates. The result is stuffiness. From Shen's studies, it was apparent that measures of nasal volume (acoustic rhinometry), nasal resistance, and 3-D reconstructions, have limited value for predicting nasal stuffiness.

As shown in FIG. 1, TRPM8 nerve fibers face the airway lumen and may be utilized to mimic negative heat flux. The nasal and sinus mucosae are abundantly lined with submucosal glands that vigorously produce serous and mucinous secretions in response to noxious agents (Widdicombe et al., 2015; Hill et al., 2022). When mucus is hypersecreted, for example, in response to viral cytokines, there is an increase in nasal cavity congestion. The term “rhinitis” applies only to the nasal cavity, but sinusitis is an equally significant source of pathology. Surprisingly, applications of cooling agonists to the mucosae of the OMC not only relieved stuffiness but also inhibited mucus hypersecretion.

### Ostiomeatal Complex and Site of Drug Delivery

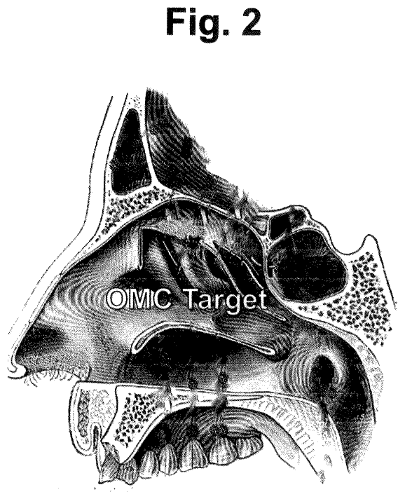

The lateral wall of the nasal cavity has three turbinates (superior, middle, and inferior) that protrude like circular shells (conchae) (FIG. 2). The space underneath the turbinates are called meatus. The middle meatus is about the size of the little finger, and the inferior meatus about the size of the index finger. The ostiomeatal complex, the principal drug target site, is located beneath the middle turbinate and has apertures (ostia) that connect the sinuses to the middle and superior meatus. On the mucosal surfaces of the nasal cavity are the nerve endings containing TRPM8 receptors. The lines from Keat's poem “To Autumn” captures the anatomic image: “ . . . the vines that round the thatch-eaves run . . . ”, the vines being the TRPM8 nerve fibers and the eaves is the middle meatus.

Liquid drops administered into the nostrils will run into the inferior meatus- and enter the nasopharynx via the choana. Manually powered sprays will deposit aerosols in the antrum and disperse on the top surface of the turbinates. To reach the OMC, the active ingredient, suspended in liquid or in solution, is best deposited into the central nasal cavity at about 0.35 to 0.5 mL per nostril with a electronic power nebulizer. A further step to aid dispersion into the OMC is to compress and release the nostrils after instillation or spraying. The automatic vigorous reflex inhalation, sniff, or snorting after compression and release helps effectively distribute the active ingredient onto the OMC. This is called the “pinch and release” method.

### Nasal Discharge: Vascular Leakage Versus Mucus Hypersecretion

The TRPM8 receptor target is an integral membrane protein on nerve fibers of the OMC. The mucosa of the OMC has a complex structure (FIG. 2). The mucosa of the nasal cavity consists of three layers: epithelium, lamina propria (submucosa), and muscularis mucosae. Epithelium is the surface layer that encounters air. The lamina propria contains blood vessels, glands, nerves, lymphatic vessels, connective tissue, and immune cells. The mucosal blood flow regulates the temperature of the inspired air. The secretions humidify the air, trap particles, and provide some immune defense against pathogens. The nerves regulate blood vessels and secretions and act as thermo- and mechano-sensors. The entire system is dynamic, sensitive, and vulnerable to injury.

The illustration by Rogers (2007) (FIG. 11) clearly shows the separate pathways of vascular leakage and mucus hypersecretion. A classic “regulatory mediator” such as histamine causes plasma exudation (vascular leakage) by opening up pores on the margins of endothelial cells in the post-capillary venules. The exudations increases the volume of fluids in the extracellular matrix and cause “congestion.” Mucus comes from a different source. The embedded submucosal glands supply serous and mucinous secretions from exocrine ducts. Serous fluids are transparent and watery and contain electrolytes and proteins, such as amylase and lysozyme. The mucus is thick, gel-like, viscous, and contains mucins (large glycoproteins visible as granules). The mucus is ˜95% water, but when dried, it forms “snot” and crusts in the nasal cavity. Mucus is also a source of nutrients for infections and, together with inflammatory exudates, form “phlegm.” For a healthy and clean nasal cavity, a subject must expel excess phlegm by blowing or swallowing. Otherwise, phlegm becomes a source of obstruction and pathology.

Airway mucus is a viscoelastic gel that shields the airway epithelium from noxious agents and facilitates their removal via mucociliary clearance (MCC). However, it is well known that viral infections of the upper respiratory tract exacerbate the signs and symptoms of patients with lower respiratory tract disease such as chronic obstructive pulmonary disease (COPD), cystic fibrosis (CF), bronchiectasis, and asthma. In such patients, mucus hypersecretion is pathogenic because the entangled mucus changes in pore size and hydration, interferes with MCC, reduces neutrophil migration, decreases pathogen capture, sustains bacterial infection; and accelerates lung function decline (Pangeni et al., Intern. J. Pharmaceu. 634:122661, 2023). Thus, a hypersecreting nasal mucosa will exacerbate the lower respiratory tract as the mucus moves down the nasopharynx and some of it enters the lower airways. A healthy respiratory mucosa throughout the airways requires an efficient MCC that is not hindered by excess fluids and mucus.

Mechanisms of “Curing” Rhinitis with a TRPM8 Agonist

The goals of the discovery are to reduce mucus hypersecretion, to reduce the sense of blocked breathing, and to promote debris removal from an inflamed nasal cavity. For the condition known as viral rhinosinusitis (e.g., the common cold), the goals are to allow the subject to breathe comfortably and have a good night's sleep. To enable this treatment the drug target sites are defined as TRPM8 receptors on neuronal endings of the OMC. Specific p-menthanecarboxamide TRPM8 agonists are delivered to the OMC with a nebulizer. A gravimetric method for quantifying anterior nasal discharge is used to show drug effectiveness, and candidates are identified for inhibiting inflammatory nasal discharge. Examples of active ingredients, delivered to the OMC, were rapid in onset, and have long-lasting benefits in rhinosinusitis.

In this discovery, the treatment strategy is to vigorously apply a TRPM8 agonist to nerve endings in the nasal cavity to create a sense of negative heat flux (cooling). The result is “cool breathing” to alleviate stuffiness. The cooling effect is stark, clear, and unmistakable, akin to stepping out into 40 to 55° F. air and taking a deep breath. Surprisingly, in patients with rhinitis, the secretion of phlegm was concurrently decreased. When the treatment was repeated for three to five days, the patient with “chronic” rhinitis viewed their disorder as being under control and “cured.” The decline in phlegm could be objectively measured. It is surprising that the primary pathological events of rhinitis, a stuffed nose, and excess phlegm can be stopped.

The analogy of the mechanism of treatment would be a wheat field (nasal mucosa) on fire (inflamed), and the dedicated TRPM8 nerve fiber acts as an “ice cream hose” to cool and stop the fire. The ice cream hose also dampens the wheat field so that the wheat field is no longer combustible, and less likely to produce debris and secretions (phlegm). The inflamed wheat field may heal if this ice cream hose is maintained. This vision of rhinitis assumes that “chronic” rhinitis is treatable as an “acute” inflammatory event, like a mosquito bite or a pustule on the nasal mucosa. If a TRPM8 agonist attenuates the “acute” event, the “chronic” condition is cured. An essential treatment strategy is to rid the nasal cavity of excess phlegm during inflammation because the accumulation of phlegm produces chronic damage. In a CT scan, the impacted phlegm illustrates impaired clearance, so the goal is to remove this phlegm, which is caused by excess mucus.

Liu et al. (Medicine 96:31, 2017) identified TRPM8 in primary cultures of human nasal epithelium and showed that these cells secreted MUC5AC (mucin) in response to cooling or l-menthol. MUC5AC is produced by goblet cells of the respiratory epithelium, whereas MUC5B is the principal mucin of the submucosal glands. The submucosal glands account for ≥85% of the mucus secreted in the nasal cavity (Widdicombe 2015). Menthol increases tearing in humans, an effect evoked by the corneal nerves. In TRPM8 knockout mice, this tearing is not seen (Robbins et al., Invest Ophthalmol Vis Sci. 53:7034-7042, 2012). Parra et al. (Nature Medicine, 16:1396-1399, 2010) also studied TRPM8 knockout mice. They concluded that receptor agonists might help promote secretions from the lacrimal gland for dry eye disease, in the oral cavity for burning mouth syndrome, and in the vaginal tract for dryness. Thus, in the papers by Liu, Robbins and Parra, the thinking is that TRPM8 activation promotes secretions from glands, whereas the findings here have the opposite conclusion: namely, TRPM8 activation inhibits mucus hypersecretion.

The molecular and cellular events envisioned are: injury→(early response) release of inflammatory mediators→activation of neuronal networks→release of mucins, proteoglycans, connective tissue proteins from glands→unfurling of polymers to expose hydrophilic groups→creation of negative interstitial fluid pressure (−Pif)→rapid movement of fluid from the vascular compartment to the interstitial space→edema and distortion of tissue architecture, congestion, hypoxia→(late response) activation of immune response and repair, release of cytokines→chronic inflammation. The TRPM8 agonists inhibit the early steps of the pathological process. Some experimental evidence for these events is discussed and in Wei (1993); Wei and Thomas (Annu. Rev. Pharmacol. Toxicol. 33:91-108, 1993); Baluk et al. and Wei (J. Pharmacol. Exp. Ther. 284:693-699,1998) Widdicombe (2015); and Wiig (2011).

### Finding Ideal Ingredient to Inhibit Mucus Hypersecretion

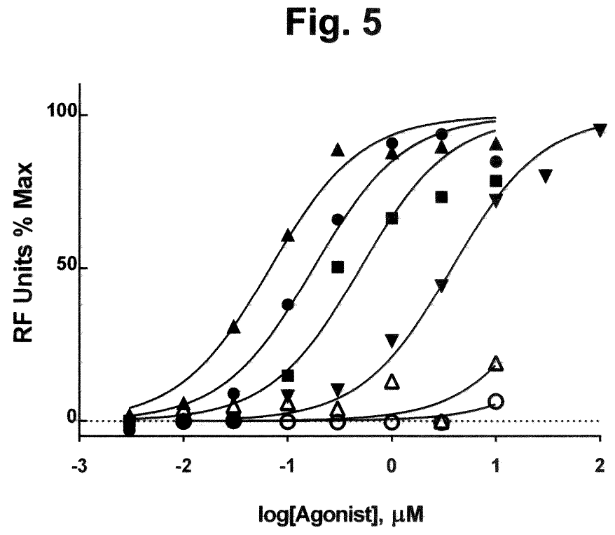

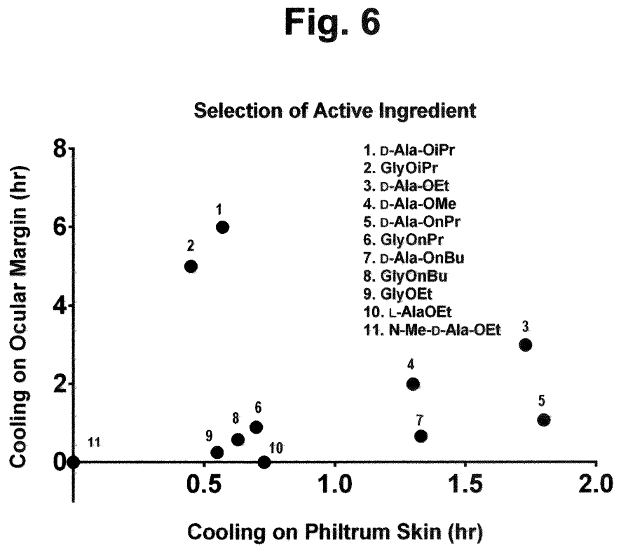

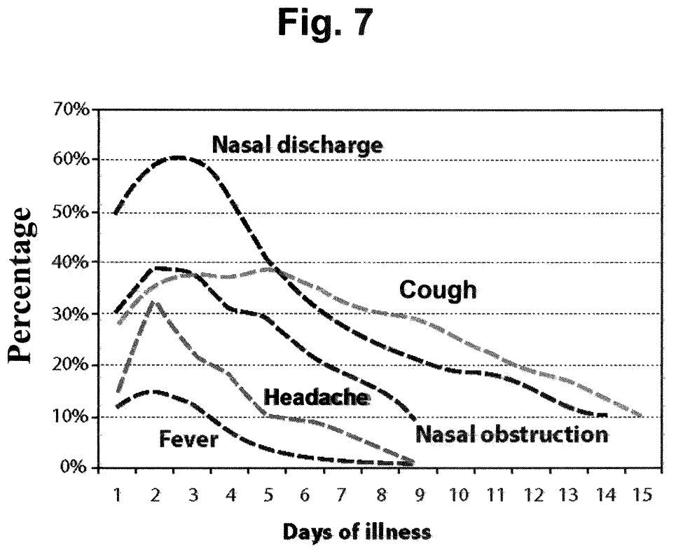

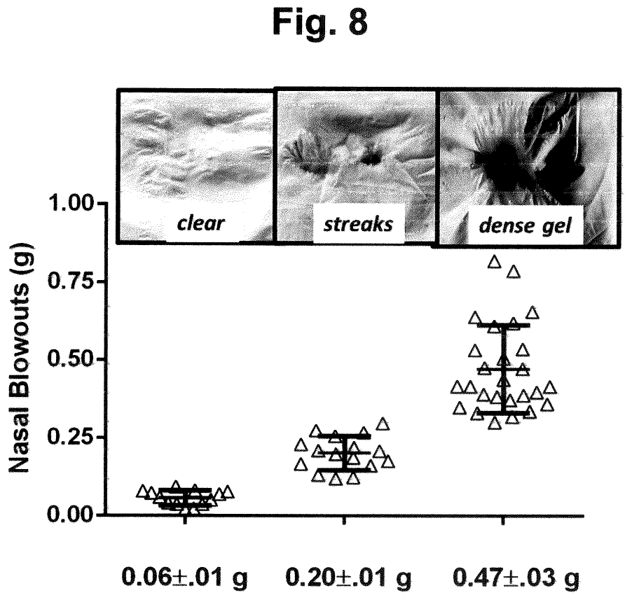

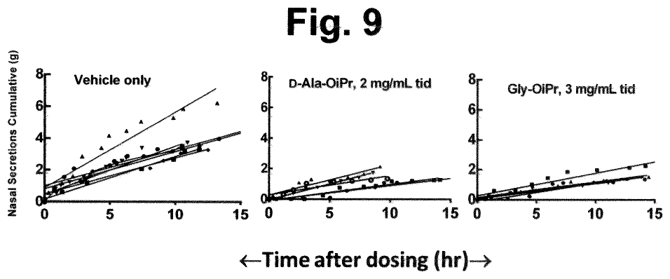

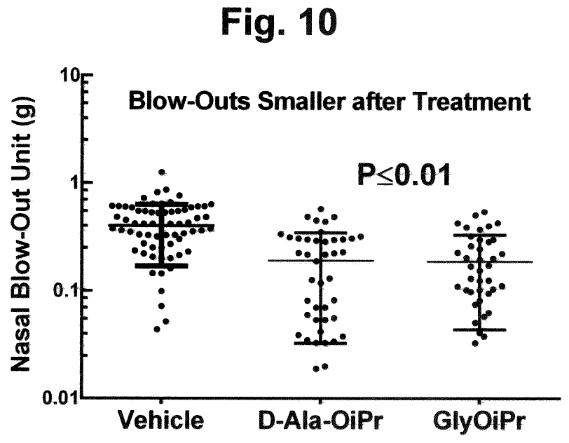

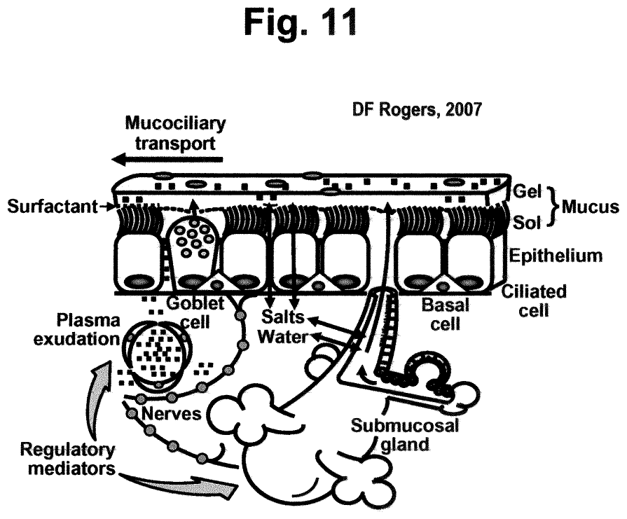

In pharmacology, “specific” and “selective” have distinct meanings. Specific refers to a drug's ability to target and interact with a particular receptor target with high affinity and efficacy. The first measure of specificity is receptor potency, usually the median effective dose (EC₅₀) for receptor activation (FIG. 4). A specific drug should bind to its intended target site with little or no affinity for other receptors. The cooling agents used in this study activate TRPM8 but not the nociceptive TRPV1 or TRPA1 (FIG. 5). Selectivity refers to a drug's ability to interact with multiple systems preferentially. A selective drug may have more extended activity, such as pain, or preferably more refreshed cooling. Selectivity is a relative effect based on physiological effects or pharmacokinetics, not receptor potency or specificity. The choice of the ideal selective drug depends on the experiment.

1 . . .

There are many TRPM8 agonists known to the art, the prototype being l-menthol. However, menthol is non-specific. It interacts with TRPA1 at a greater potency than TRPM8 (Karashima et al., J. Neurosci. 27:9874-9884, 2007). Moreover, menthol stimulates MUC5AC from goblet cells of human nasal epithelium (Liu et al., 2017). Examples of TRPM8 agonists are in FIG. 4, Table 4, and their EC₅₀ values are in Table 5. Further evidence of specificity is shown with GlyOiPr in FIG. 5. GlyOiPr activates TRPM8 but not TRPV1 or TRPA1. D-Ala-OiPr also has this selectivity. The EC₅₀ is the first step in choosing a lead candidate. Many closely related analogs have overlapping 95% confidence levels for their EC₅₀ value, and the EC₅₀ will not predict the duration of drug action at target sites, ease of delivery to target, side effects such as taste aversion, or other adverse events. To make the right choice, It is necessary to conduct experiments to measure “selectivity.”

### Chemical Synthesis and Structure Activity Relationships

Menthol is a C₁₀H₂₀O terpenoid alcohol (MW 156.27) with three chiral centers leading to eight possible stereoisomers. The l-menthol isomer is the principal cooling isomer. The alkyl-substituted cyclohexane ring of menthol is called p-menthane and the carboxylic acid derivative of p-menthane, called WS-1, is the standard precursor for the synthesis of p-menthanecarboxamides (Table 2). The preferred embodiment isomers have the steric configuration of l-menthol, namely, (1R,2S,5R)-2-isopropyl-5-methyl-cyclohexanol (see Table 2, 10).

The discovery of p-menthanecarboxamide cooling compounds by Wilkinson Sword scientists 45+ years ago (Watson et al., 1978) changed the world of oral care. By recognizing that the hydrogen bonding functional group (carbonyl), when attached to a compact hydrocarbon skeleton (cyclohexane), was essential for cooling activity, these scientists led the way for developing cooling p-menthanecarboxamides that are now widely used in mouth wash, toothpaste and chewing gum. Recent studies have identified the likely molecular binding pockets and anchoring sites for p-menthanecarboxamides when activating the TRPM8 receptor. These studies were conducted by cryoelectron microscopy at a resolution of ≤4 Å (Yin and Lee, 2020).

The bioassay used by Watson et al. (1978) for cooling relied on filter papers impregnated with test substances and placed on the tongue's surface. However, the subtleties of SAR on cooling thresholds at tissue sites other than the oral cavity remain unknown. For instance, for a series of p-menthanecarboxamides, cooling in the oral cavity has not been rigorously compared to cooling on the skin, the pharyngeal surface, the ocular surface, the genitourinary tract, or the nasal cavity. It's important to note that the activity on one tissue does not necessarily extrapolate to a different site. For instance, an agent can cause pain and rhinorrhea in the nasal cavity, but these effects will not occur in the mouth.

In SAR studies of p-menthanecarboxamides for the nasal cavity, three key attributes are essential for an effective agent:
a. A rapid and refreshing cooling sensation that relieves nasal stuffiness and stops mucus hypersecretion. These target effects will treat viral acute rhinosinusitis. Agents especially effective for these endpoints are D-Ala-OiPr, D-Ala-OnPr, Gly-OiPr, and Gly-OnPr (see Table 10 for structures).
b. A slow onset, long-lasting cooling action that reduces mucus hypersecretion and avoids mouth breathing. Examples are WS-12, FEMEA 4549, and FEMA 4864.
c. An agent that combines fast onset with a long duration of action, such as CPS-125, will be ideal for chronic rhinosinusitis.

In summary, the discovery of p-menthanecarboxamide cooling compounds led to applications in oral care and skin. Further research and development could lead to the discovery of more effective treatments for other tissue sites as well. The potential of these compounds to improve nasal cavity function and breathing is described here. The technical insights into applications were aided by using a top-loading balance for measuring mucus hypersecretion and an electronically-powered nebulizer for delivery of the p-menthanecarboxamide to the ostiomeatal complex.

In one approach, alkyl esters of the D-alanine or glycine amide of the carboxylic acid corresponding to l-menthol may be prepared by reacting the acid halide (e.g., the acid chloride) of the carboxylic acid corresponding to l-menthol with the appropriate glycine alkyl ester, for example, as illustrated below for the glycine amide. Many glycine alkyl esters (including methyl and ethyl glycine esters) are commercially available from Sigma-Aldrich. Other amino acid alkyl esters may be prepared by those of ordinary skill in the art using known and conventional methods. For example, the ethyl ester compound (where R is Et), referred to herein as GlyOEt, was prepared as follows: 1.0 g of glycine ethyl ester hydrochloride (Sigma-Aldrich) was dissolved in 28 mL diethyl ether and 1 mL double-distilled water and cooled to 0° C. A pinch of the catalyst diaminopyrimidine was added. 1.62 mL of p-menthoyl chloride was added dropwise, followed by 2 ml of triethylamine. Clumps of white precipitate appeared in the mixture, which was stirred overnight at room temperature. The precipitate was collected, dissolved in ethyl acetate, washed with double-distilled water, and dried over sodium sulfate. The organic phase, evaporated under reduced pressure, yielded the final product (2 g), which crystallized at room temperature and was of high purity.

High-performance liquid chromatography (HPLC) revealed a single peak accounting for more than 95% of the material. Mass spectroscopy (MS) and nuclear magnetic resonance (NMR) confirmed the identity. The isopropyl compound (where R is iPr), referred to herein as GlyOiPr, was prepared by Phoenix Pharmaceuticals, Burlingame, California (www.phoenixpeptide.com): Lot No. 427497; C₁₆H₂₉NO₃; molecular weight 283.41; purity >95% by weight; appearance: white crystalline powder. Additional compounds were synthesized by Diapharm Ltd., St. Petersburg, Russia.

### Specificity for TRPM8 Receptor, EC50 Values.

The drug target for this invention is the TRPM8 receptor on nerve fibers that innervate the mucous membranes of the ostiometal complex. The respiratory epithelium and mucosa is thin and non-keratinized. As shown in the photomicrograph (FIG. 1), the TRPM8 nerve fiber is on the surface on the mucosa of the membranes facing the airway lumen. Activation of the TRPM8 receptor opens voltage-gated ion channels followed by depolarization of the neuronal membrane. The afferent signals mimic the effect of heat abstraction from the membrane surface and convey the sense of coolness to the brain from that location.

TABLE 1
Inventive Steps for Testing Preferred Embodiments

| Parameters | | Experimental Rationale |
|---|---|---|
| 1. | accessibility to target | identifying OMC as target, choosing method of delivery, |
| | | sniffing or snorting to enhance accessibility to OMC |
| 2. | penetration of drug through | chose hydrophilic embodiments, use of electronically |
| | the secreted nasal fluids | assisted nebulizer to reach target in ostiomeatal complex |
| 3. | penetration to receptor at | selecting specific TRPM8 agonists ≤300 Daltons, |
| | target after delivery | evaluating octanol/water partition coefficient |
| 4. | retention of drug at target | diisopropyl legs and isopropyl esters to enhance |
| | | refreshing cooling effect and retention for longer |
| | | duration of action |
| | | testing p-menthanecarboxamide with aryl group that |
| | | may enhance retention (WS-12) |
| 5. | avoid penetration into | icilin or dialkylphosphorylalkanes are avoided because of |
| | systemic circulation | systemic effects |
| 6. | identify optimal sensations. | refreshed cooling with rapid onset, absence of dry |
| 7. | Choosing clinical conditions: | mouth upon awakening. |
| | e.g. viral ARC, CRS, or | gravimetric method for measuring nasal discharge, |
| | allergic and non-allergic | quantification allows proof of efficacy |
| | rhinitis | |

The treatment objective in this discovery is to vigorously deliver a TRPM8 agonist to nerve endings in the ostiomeatal complex to create a sense of negative heat flux (cooling). This results in “cool breathing” to alleviate stuffiness. The cooling effect is stark, clear, and unmistakable: it is like stepping out into 40 to 55° F. air and taking a deep breath.

The applicant has synthesized and examined a series of p-menthanecarboxamides conjugated to amino acid esters. These studies, described in Wei U.S. Pat. Nos. 8,426,463, 8,476,317, and 8,853,267, are incorporated herein by reference. This class of molecules is called N-alkylcarbonyl-amino acid esters. GlyOiPr is a p-menthanecarboxamide conjugate of glycine isopropyl (iPr) ester. It is similar in structure to WS-5, a cooling agent used as a food additive. WS-5 is the p-menthane-glycine ethyl (Et) ester, or GlyOEt. GlyOiPr has a molecular weight of 283.4 Daltons. It is an odorless, white crystalline solid, stable at room temperature and physiological pH. In the oral cavity, the sensory qualities of GlyOiPr differ from WS-5, being more refreshing and less bitter than WS-5 (Johnson et al., 2018). Table 4 shows examples of compounds prepared and tested for TRPM8 receptor activation. These in vitro effects were measured in CHO cells transfected with the hTRPM8 channel. Receptor activity was quantified using a Fluo-8 calcium kit and a Fluorescence Imaging Plate Reader (FLIPRTETRA™). The assays were conducted by ChanTest Corporation, 14656 Neo Parkway, Cleveland, OH 44128. As an example of receptor specificity, GlyOiPr and D-Ala-OiPr was tested on TRPV1 and TRPA1 (FIG. 5) and shown to be specific for TRPM8. Capsaicin and mustard oil are positive controls for the TRPV1 and TRPA1 assays. D-Ala-OiPr is also specific for TRPM8 and inactive on TRV1 and TRPA1 (data not shown).

TABLE 2
p-Menthanecarboxamides, Structure-Activities

| | | |
|---|---|---|
| 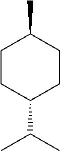 | 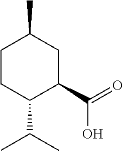 | 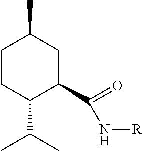 |
| p-menthane | p-methane carboxylic acid | p-menthanecarboxamide** |
| | (WS-1)* | |

| | |
|---|---|
| WS-1 has the steric orientation of I-menthol: (1R,2S,5R)-configuration. For D-Ala and D-Gly analogs, the R substituent is an amino acid conjugate. For aryl-substitutents, when R,R′ are hydrogen the compound is more lipophilic and difficult to deliver to OMC. When —z is made more polar, delivery is improved: for example when z = sulfamoyl. | 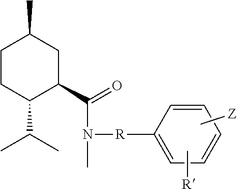 |

*there are three chiral centers and hence eight enantiomers.
**by convention, p-menthanecarboxamide is spelled as one word.

In the case of intranasal (INC) sprays, the airflow rate is determined by the inhalation speed and the spray velocity caused by the actuation force. The patient's actuation force of the spray device determines the distribution pattern in the nasal cavity. Preliminary steps include nose blowing, nose rinsing, head positioning, spray positioning; depth of the spray into the nostril, and closing of the contralateral nostril. How each of these steps affect particle deposition is not known. Predicting the correct deposition based on plume geometry is challenging because of the complex anatomy of the nasal passage. It is know that larger droplets are lost by inertial impact at the entrance of the nose.

The EC₅₀ has limited value in predicting in vivo activity (FIG. 4). Although the p-menthane Gly-esters and D-Ala-esters are excellent cooling agents, they show a remarkable heterogeneity of responses in various tissues. The OiPr analogs act longer on the ocular margin when compared to the effects on the philtrum skin (FIG. 6, Table 6,7). The D-Ala-OiPr is the longest-acting, and GlyOiPr is No. 2 on the eyelid margin. These two compounds are surprising “outliers” favoring non-keratinized membranes. GlyOEt (WS-5), similar in structure to GlyOiPr, is much less active on the ocular margin (no. 9 vs. no. 2), although the EC₅₀ values overlap. These compounds' relative activity on nasal membranes has yet to be systematically explored. The automated nebulizer permits a detailed study of dose response and comparisons of analogs on the nasal membranes.

The prolonged effects of D-Ala-OiPr and GlyOiPr on the ocular margins were surprising and unexpected and lasted, on average, for 5 hours. Increasing the test concentration of GlyOiPr from 1 mg/mL to 1.5 mg/mL further increased the duration on the ocular surface to about 6.5 hours. By comparison, increasing the concentration of GlyOEt from 1 mg/ml to 2 mg/ml or even to 5 mg/ml increased the cooling intensity by only 15 to 22 minutes, respectively. The selective actions of GlyOiPr and D-Ala-OiPr on the ocular margins are unusual. Further differences are seen on the oropharyngeal surface where both GlyOiPr and GlyOEt exerted a refreshing cooling sensation but GlyOiPr unlike either GlyOEt or GlyOnPr has less bitter off-taste (Johnson et al, 2018, vide supra). On the eyelids, the duration of action GlyOiPr >>GlyOnPr >GlyOEt. Thus, the OiPr analogs have surprising and unexpected properties that are distinct from -OMe, -OEt, -OnPr, and -OnBu analogs.

### Tests for Selectivity: Bioassay Endpoints for Nasal Inflammation

Cooling agents have been assayed in various ways depending on intended use. For example, for the skin the test substance can be applied on philtrum skin, using descriptions of coolness on a scale of 0 to 3. For the ocular surface, the active ingredient can be applied to the closed eyelids using a wipe. In the oral cavity, drug applications are on the posterior dorsal surface of the tongue. For the oropharyngeal surface, the cooling agent may be applied as an orally disintegrating tablet. The ability of a cooling agent to antagonize a noxious stimuli may also be tested, using nociception from citric acid or chili pepper sauce. Responses are usually measured using a Visual Analog Scale with a numerical rating. For nasal responses, however, such procedures have not been standardized. Some endpoints to consider are open and comfortable breathing, the presence or absence of sniffles, and the secretion of phlegm.

The nasal cavity mucosa is a complex system. When there is inflammation (rhinitis), several components are activated and sentient.
The perception of impaired breathing can be evoked by pinching the nasal ala, the outer, fleshy, and cartilaginous structure that forms the wing-like sides of the nostrils. Pinching is a physical method to produce the sensation of stuffiness. A heat load in the nasal cavity will also cause stuffiness. For example, a facemask in hot weather will cause a heat load and stuffiness. Stuffiness is relieved by a negative heat flux. Movement of cold air across the nasal cavity surface, e.g., with an air conditioner, will relieve stuffiness. Stimulation of the TRPM8 receptor on nerve fibers with an agonist mediates coolness via a similar mechanism. Stuffiness is the main complaint of patients with rhinitis because this sensation at night will interfere with sleep.
Mucus is secreted from glands of the nasal mucosa. Mucus is a transparent white protein when secreted but when mixed with exudates and cell debris it adopts a slimly, viscous yellow gel called phlegm or gunk. When copious, It is dense and unpleasant to look at when blown out onto a wipe. Nasal discharge is a primary sign of the common cold (FIG. 7). When phlegm dries, it becomes crusted and hardened, and called “snot.” Phlegm can physically reduce nasal cavity volume, but the patient with rhinitis typically clears the mucus by blowing the nose or using irrigation with water or saline. The phlegm in the nasosinus cavity when extensive, give the opacity that appears in a CT scan. The weight of phlegm blown out onto a tissue paper provides a quantitative and dynamic index of nasal inflammation.
Irritation of the nasal cavity may cause a watery, runny nose. These secretions are thin, and if blown on a wipe, it is a clear liquid. The liquid source may be from tears draining into the nasal cavity via the nasolacrimal duct, fluids secreted from serous glands and under cholinergic control, or plasma leakage from dilated capillaries, for example, after histamine release. The condition of “sniffles” occurs after intense cold exposure, such as a “skier's nose.” A subject may snort the accumulated liquid, and this gurgling sound is sometimes interpreted as “congestion.” This symptom causes minor symptoms because there is no associated discomfort or blockage. A muscarinic antagonist such ipratropium is sometimes used for the common cold when the watery secretions because excessive, sometimes up to 5 g per hour.

TABLE 3
Treatment goals of nasal cavity inflammation (↑).

| Cool Breathing | | Mucus/Phlegm | | Sniffles | |
|---|---|---|---|---|---|
| Normal | | Normal | | Normal | |
| Partially Stuffed | ↑ | Some | ↑ | Some liquid | ↑ |
| Fully Stuffed | ↑ | Plenty | ↑ | Lots of liquid | ↑ |

The basic goal of treating nasal inflammation is to achieve the goals shown in Table 1 and 3. The applicant finds delivery of the preferred embodiments into the nasal cavity; using a penetrating nebulizer, relieves all three components of nasal dysfunction, and has long-term benefits. Control of the perception of stuffiness is complete. Surprisingly, phlegm production was also reduced. The change in parameter of nasal inflammation by a TRPM8 agonist was dramatic and spectacular (FIG. 8, FIG. 9, FIG. 10). The effects on sniffles were less obvious as the occurrence of this symptom was not consistently observed. A 3 to 5-day course treatment with three doses daily enabled patient comfort and essentially free of symptoms and signs of rhinitis.

TABLE 4
Chemical identities of some TRPM8 agonists

| Name | Chemical Name | Chemical Structure: |
|---|---|---|
| I-menthol | (1R,2S,5R)-2-isopropyl-5-methyl- cyclohexanol [(−)-menthol] | 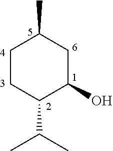 |
| icilin | 1-[2-hydroxy]-4-[3-nitrophenyl]-1,2,3,6- tetrahydropyrimidine-2-one [icilin, AG-3- 5,3,6-Dihydro-1-(2-hdyroxyphenyl)-4- (3-nitrophenyl)-2(1H)-pyrimidinone] | 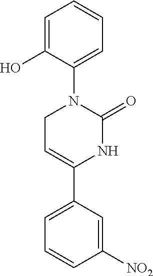 [SMILES: O=C1NC(c2cccc([N+](=O)[O-])c2)=CCN1c1ccccc1O] |
| D-Ala-OiPr | (R)-2-[((1R,2S,5R)-2-isopropyl- 5-methyl-cyclohexanecarbonyl)amino]- propionic acid i-propyl ester | 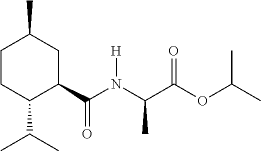 [SMILES: CC(C)OC(=O)[C@@H](C)NC(=O)[C@@H]1C[C@H](C)CC[C@H]1C(C)C] |
| D-Ala-OEt | (R)-2-[((1R,2S,5R)-2-isopropyl- 5-methyl-cyclohexanecarbonyl)-amino]- propionic acid ethyl ester | 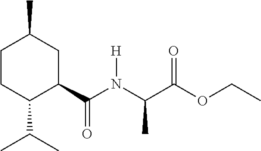 [SMILES: CCOC(=O)[C@@H](C)NC(=O)[C@@H]1C[C@H](C)CC[C@H]1C(C)C] |
| L-Ala-OEt | (S)-2-[((1R,2S,5R)-2-isopropyl- 5-methyl-cyclohexanecarbonyl)-amino]- propionic acid ethyl ester | 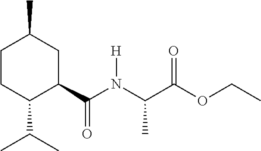 [SMILES: CCOC(=O)[C@H](C)NC(=O)[C@@H]1C[C@H](C)CC[C@H]1C(C)C] |
| D-Ala-OnPr | (R)-2-[((1R,2S,5R)-2-isopropyl- 5-methyl-cyclohexanecarbonyl)-amino]- propionic acid n-propyl ester | 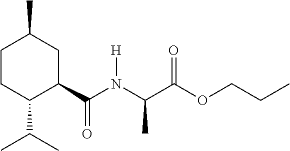 [SMILES: CCCOC(=O)[C@@H](C)NC(=O)[C@@H]1C[C@H](C)CC[C@H]1C(C)C] |
| GlyOEt | [((1R,2S,5R)-2-isopropyl- 5-methyl-cyclohexanecarbonyl)- amino]-acetic acid ethyl ester | 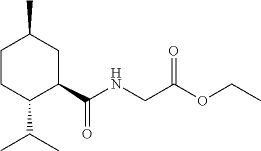 [SMILES: CCOC(=O)CNC(=O)[C@@H]1C[C@H](C)CC[C@H]1C(C)C] |
| GlyOnPr | [((1R,2S,5R)-2-isopropyl- 5-methyl-cyclohexanecarbonyl)- amino]-acetic acid n-propyl ester | 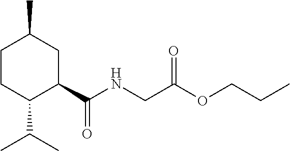 [SMILES: CCCOC(=O)CNC(=O)[C@@H]1C[C@H](C)CC[C@H]1C(C)C] |
| GlyOiPr | [((1R,2S,5R)-2-isopropyl- 5-methyl-cyclohexanecarbonyl)- amino]-acetic acid isopropyl ester | 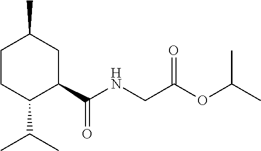 [SMILES: CC(C)OC(=O)CNC(=O)[C@@H]1C[C@H](C)CC[C@H]1C(C)C] |
| GlyOnBu | [((1R,2S,5R)-2-isopropyl- 5-methyl-cyclohexanecarbonyl)- amino]-acetic acid n-butyl ester | 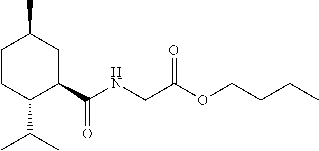 [SMILES: CCCCOC(=O)CNC(=O)[C@@H]1C[C@H](C)CC[C@H]1C(C)C] |

TABLE 5
Agonist activities on TRPM8 receptor assay, EC50

| | EC50 | 95% Confidence | Potency Relative |
|---|---|---|---|
| Chemical | μM | Interval | to L-menthol |
| Menthol | 3.8 | 2.5 to 5.6 | 1.0 |
| Gly-OMe | 0.50 | 0.37 to 0.90 | 7.6 |
| Gly-QEt | 0.36 | 0.30 to 0.75 | 10.6 |
| Gly-OiPr | 0.50 | 0.27 to 0.85 | 7.6 |
| Gly-OnBu | 1.36 | 0.5 to 2.1 | 2.8 |
| D-Ala-OMe | 0.59 | 0.27 to 0.75 | 6.4 |
| D-Ala-OEt | 0.27 | 0.17 to 0.42 | 14.0 |
| D-Ala-OiPr | 0.26 | 0.16 to 0.41 | 14.6 |
| D-Ala-OnBu | 1.03 | 0.4 to 2.0 | 3.7 |
| L-Ala-OEt | 24 | 17 to 34 | 0.2 |

### Experimental Procedures: Formulation and Delivery

The preferred embodiments, p-menthanecarboxamides, are relatively water-insoluble compounds and therefore delivered with the nebulizer as a homogeneous liquid suspension, although some of the solids are in solution. The active concentrations are in the range of 1 to 10 mg/mL and the use of standard solvent excipients such as ethanol and Polysorbate 80 are adequate. Addition of other excipients such as 1,2-propanediol and hyaluronic acid are acceptable. Formulation specialists are familiar with the suspension of insoluble compounds such as fluticasone and beclomethasone which are used for nasal sprayers. Hence, there is a bountiful prior art on how to make formulations of insoluble compounds for intranasal delivery. An example of the formulated preferred embodiments is shown in Table 8.

Formulations for nebulization with NasoNeb® used a standard recipe of (Table 8): 1% of the active ingredient (1 to 10 mg/mL), 2% Tween 80 (Polysorbate 80), 1 to 5% ethanol, and made up to 100% with water. The mixture is centrifuged in a FlackTek, Inc., Speedmixer for at least 10 min at 3000 rpm. The Speedmixer spins the sample in 3 dimensions. The result is a white homogeneous liquid suspension with some of the solids now dissolved. For dosing, ˜2 to 3 mL is loaded into the canister of the nebulizer. The nebulizer nozzle tip is placed in the nostril and activated by pressing a button for 3 to 5 sec using a clock timer. The delivered volume was remarkably consistent at for a 3-sec and 5-sec activation resulted in 0.62±0.1 mL and 0.73±0.1 mL delivered per nostril, respectively. For D-Ala-OiPr a 5-sec activation will deliver 2*0.73 mL*2 mg/ml, or a total dose of 3 mg per nose. The nasal cavity is relatively tolerant to exogenous agents with irritant properties, as many rinses are used for rhinitis and sinusitis. For some p-menthanecarboxamide analogs, a 2% (20 mg/mL) solution is possible and contemplated.

TABLE 6
Bioassay on Philtrum, Ocular Margins, and Oral Cavity

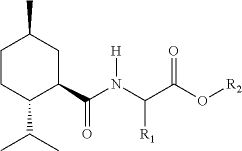

| | | | Philtrum | Eyelids/Ocular | |
|---|---|---|---|---|---|
| | | | Skin | Surface | Chili-Pepper |
| Compounds | R₁ | R₂ | (min) | (min) | Sauce Test |
| Gly-OEt | H | Et | 24 | 15 | 0 |
| (WS-5) | | | | | |
| Gly-OnPr | H | nPr | 42 | 54 | 0 |
| Gly-OnBu | H | nBu | 38 | 35 | 0 |
| D-Ala-OMe | Me | Me | 77 | 120 | ++ |
| D-Ala-OEt | Me | Et | 103 | 180 | ++ |
| D-Ala-OiPr | Me | iPr | 34 | 360 | ++ |
| D-Ala-OnPr | Me | nPr | 108 | 65 | + |
| D-Ala-OnBu | Me | nBu | 80 | 40 | 0 |
| L-Ala-OMe | Me | Et | 25 | 10 | 0 |
| L-Ala-OEt | Me | Et | 34 | 0 | 0 |
| L-Ala-OiPr | Me | Et | 27 | 15 | 0 |
| L-Ala-OnPr | Me | Et | 22 | 10 | 0 |
| L-Ala-OnBu | Me | Et | 0 | 0 | 0 |
| D-NMeAla-OEt | Me | Me | 0 | 0 | 0 |

TABLE 7
Bioassay of Gly Esters on Philtrum, Ocular Margins.

| | | |
|---|---|---|
| | Philtrum Skin | |
| Code | (min) ⁽¹⁾ | Ocular Surface (min) ⁽²⁾ |

| | | | |
|---|---|---|---|
| GlyOMe | 10 | 5 | Oropharyngeal |
| | | | Surface ⁽³⁾ |
| GlyOEt | 24 | 15 | 0 |
| GlyOnPr | 42 | 54 | 0 |
| GlyOiPr | 27 | 300 | ++ |
| GlyOnBu | 38 | 35 | 0 |

The delivered volume from the nebulizer is determined by pressing the pump button for a fixed time. In the current method, a 3-sec and 5-sec activation resulted in 0.62±0.1 mL and 0.73±0.1 mL delivered per nostril, respectively. Thus, for a 5 mg/mL suspension, the dose is (0.73*5*2=) 7.3 mg per nose. For a 5-sec delivery, the procedure should be viewed as a nasal rinse (synonym: nasal lavage or nasal irrigation) because there is some overflow and dripping of the solution out of the nose. This is desirable, as the goal is to rinse the surface of the nasal cavity and not use the delivery as a point source of drug absorption. The total target surface of the nasal cavity is about the area of one side of the hand. The goal is to cover much of this surface area with the preferred embodiment in liquid form. An overflow of a few drops from the nose is acceptable.

TABLE 8
Formulation of cooling ingredient for nebulization.

| | g/100 | |
|---|---|---|
| Substance | ml | |
| Active | 0.1 to | Structures shown in Table 4. |
| Ingredient | 1% | |
| Tween 80 | 1 to 2% | Tween 80, also known as Polysorbate 80, is a |
| | | commonly used nonionic surfactant and emulsifier |
| | | in pharmaceutical formulations. It helps disperse |
| | | poorly water-soluble drugs evenly in an aqueous |
| | | suspension. |
| Ethanol | 1 to 5% | Ethanol is added to aid solubility and to reduce the |
| | | surface tension of the liquid in its container. |
| Water | 92-95% | Homogeneous liquid suspension after hi-speed |
| | | centrifugation. |
| total | 100 | |

### Choosing Ingredients for Nebulizer Studies

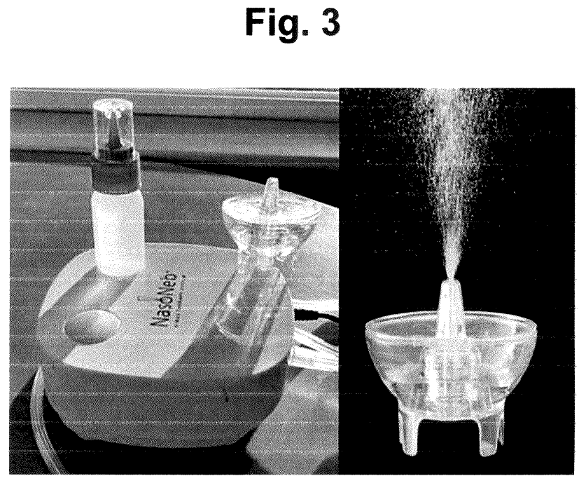

An ideal agent reproduces a comfortable cooling or sensations of negative heat flux in the nasal cavity. Negative heat reflux is when heat energy transfers away from a location. Heat flux refers to the rate of heat transfer per unit area. The sinonasal cavity mucosa differs from philtrum skin and ocular margins in histology, so the choice of the right molecule must be made by experiment. The TRPM8 agonists were chosen for screening using a nebulizer for delivery. The NasoNeb system is powerful and effective for the OMC (FIG. 3). The delivery time per nostril in three to five seconds. The results showed that the ideal analogs for treating stuffiness and for inhibiting phlegm secretion (Table 10).

These parameters were estimated in the initial trials:
Onset and offset of cooling sensations in the nasal cavity.
The intensity and quality of the cooling sensations in the nasal cavity.
Presence of effects such as sneezing or cough after dosing.
Degree of nasal stuffiness on a scale of 0 to 5.
Degree of rhinorrhea and phlegm in nasal secretions.
Questions regarding comfort and sleep at night.

Based on these results, four lead candidates were chosen for detailed examination.

### Results of Nasal Stuffiness Used Three Times a Day.

Eight subjects with rhinitis and rhinosinusitis volunteered to test cooling agents delivered with the NasoNeb® nebulizer (FIG. 3). The results were clear, robust, and evident. No statistical analysis was necessary. In a 3 to 5-day course of nebulization t.i.d., all subjects reported the ability to manage and control stuffed-up breathing in their activities. All reported comfortable sleep. The sounds of sniffling and congestion of the nose (“sniffles”), snorting, or gurgling were still present, but to a lesser degree. The subject felt it was easier to blow out liquids from the nasal cavity onto a tissue, and the volume and frequency of blowing decreased. Viscous yellow mucus was less in volume and density. The number of wipes deposited daily into a waste basket decreased by >50%. The subjects-were cheerful and enthusiastic because nasal discomfort was under control. As one subject remarked: “The stuffed-up nose problem is now history!” Severat of these cases are reported below as “Case Studies.”

TABLE 9
Representative TRPM8 agonists not chosen for further experiment.

| | | |
|---|---|---|
| I-menthol (1R,2S,5R)-2-isopropyl-5-methyl- cyclohexanol [(−)-menthol] FEMA 2665, cas 2216-51-5 | 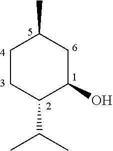 | Strong cool, clear breathing. Eye irritant, causes rhinorrehea, short cooling duration ~7 min, has harsh sensory effect. Pungency causes undesirable rhinorrhea. |
| icilin 1-[2-hydroxy]-4-[3-nitrophenyl]- 1,2,3,6-tetrahydropyrimidine-2- one CAS 36945-98-9 | 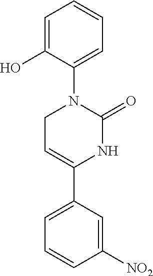 [SMILES: O=C1NC(c2cccc([N+](=O)[O-])c2)=CCN1c1ccccc1O] | Difficult to form a homogeneous suspension, causes systemic cooling at higher doses |
| D-Ala-OEt (R)-2-[((1R,2S,5R)-2-isopropyl- 5-methyl-cyclohexanecarbonyl)- amino]-propionic acid ethyl ester | 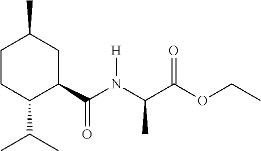 [SMILES: CCOC(=O)[C@@H](C)NC(=O)[C@@H]1C[C@H](C)CC[C@H]1C(C)C] | Bitter taste, causes coughing, shorter- acting ~15 min |
| L-Ala-OEt (S)-2-[((1R,2S,5R)-2-isopropyl- 5-methyl-cyclohexanecarbonyl)- amino]-propionic acid ethyl ester | 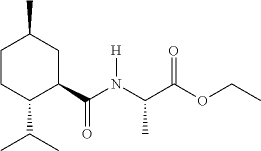 [SMILES: CCOC(=O)[C@H](C)NC(=O)[C@@H]1C[C@H](C)CC[C@H]1C(C)C] | Very little cooling, low potency on TRPM8, good only as negative control. |

Other compounds contemplated for testing include WS-30, D-3263 HCl (Dendreon), M8-Ag (Patel et al., 2014), and other molecules described in patents by ET Wei. (U.S. 8,426,463, U.S. 8,476,317, and 8,853,267)

TABLE 10
Representative TRPM8 agonists chosen for development.

| | | |
|---|---|---|
| GlyOnPr [((1R,2S,5R)-2-isopropyl- 5-methyl-cyclohexanecarbonyl)- amino]-acetic acid n-propyl ester | 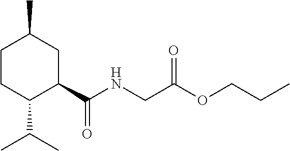 [SMILES: CCCOC(=O)CNC(=O)[C@@H]1C[C@H](C)CC[C@H]1C(C)C] | About the same duration of action as GlyOiPr, but less refreshing. |
| GlyOiPr [((1R,2S,5R)-2-isopropyl- 5-methyl-cyclohexanecarbonyl)- amino]-acetic acid isopropyl ester | 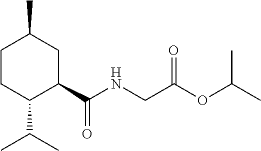 [SMILES: CC(C)OC(=O)CNC(=O)[C@@H]1C[C@H](C)CC[C@H]1C(C)C] | The sense of coolness is pure, without any harsh tones. |
| D-Ala-OnPr (R)-2-[((1R,2S,5R)-2-isopropyl- 5-methyl-cyclohexanecarbonyl)- amino]-propionic acid n-propyl ester | 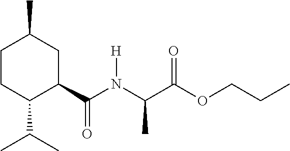 [SMILES: CCCOC(=O)[C@@H](C)NC(=O)[C@@H]1C[C@H](C)CC[C@H]1C(C)C] | Strong penetrating cool at 3 mg/mL. Excellent for viral ARS. |
| D-Ala-OiPr (R)-2-[((1R,2S,5R)-2-isopropyl- 5-methyl-cyclohexanecarbonyl)-amino]- propionic acid i-propyl ester | 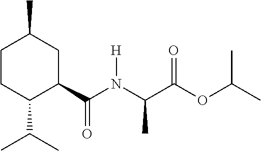 [SMILES: CC(C)OC(=O)[C@@H](C)NC(=O)[C@@H]1C[C@H](C)CC[C@H]1C(C)C] | Longer-acting than GlyOiPr. Excellent agent. It may have a slightly bitter taste. |
| CPS-125 (1R,2S,5R)-2-Isopropyl-5-methyl- cyclohexanecarboxylic acid [4- (pyrimidin-2-ylsulfamoyl)-phenyl]-amide | 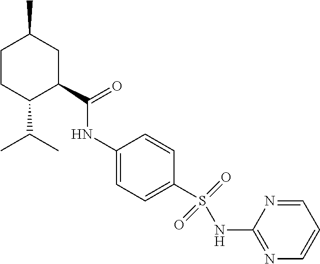 [SMILES: CC(C)[C@@H]1CC[C@@H](C)C[C@H]1C(=O)Nc1ccc(S(=O)(=O)Nc2ncccn2)cc1] | See Case Study 5. |
| WS-12 (1R,2S,5R)-2-isopropyl-N-(4- methoxyphenyl)-5-methyl- cyclohexanecarboxamide FEMA 4681, CAS 68489-09-8 | 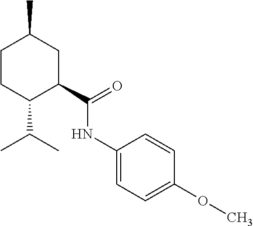 [SMILES: COc1ccc(NC(=O)[C@@H]2C[C@H](C)CC[C@H]2C(C)C)cc1] | See Case Study 5. Slow onset of cooling at 10 mg/mL. But effective for a dry mouth. |
| FEMA 4549 (1R,2S,5R)-N-(2-(pyridin-2-yl)ethyl)-3- p-menthanecarboxamide CAS 847565-09-7 | 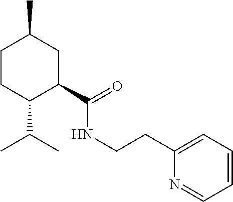 [SMILES: CC(C)[C@@H]1CC[C@@H](C)C[C@H]1C(=O)NCCc1ccccn1] | Although potent on TRPM8 receptor, slow onset and absence of refreshing cooling. |
| FEMA 4684 (1,R,2S,5R)-N-(4-(2-amino-2- oxoethyl)phenyl)-5-methyl-2-(Propan-2- yl)cyclohexanelcarboxamide CAS 1119711-29-3 | 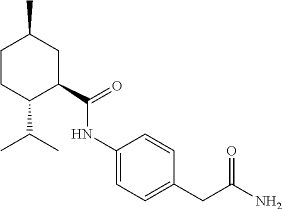 [SMILES: CC(C)[C@@H]1CC[C@@H](C)C[C@H]1C(=O)Nc1ccc(CC(N)=O)cc1] | Reported to cool the oral cavity at 1 to 10 ppm in water. |

### Unexpected Discovery: Inhibitory Effect on Mucus Secretion

The nasal cavity must be kept clean, especially from deleterious phlegm or gunk, which is a mixture of excess mucus, secretions, and debris of inflammation. Rhinologists know that phlegm harms the nasal passages, and irrigation of the nose to remove phlegm is now familiar. Stopping stuffiness in rhinitis alone has value because it is comfortable. The phlegm is the principal pathological feature. For inflamed nasal passages, a rapid and efficient medication to remove phlegm can revolutionize rhinitis treatment. Approximately 250,000 surgeries are performed each year by rhinologists (in the USA). With new cryosurgical and electrolytic ablation of nasal nerves, surgery is likely to increase, but at about $25,000 per procedure. A simple 3- to 5-day course of nebulized cooling agents to treat phlegm secretion, the worst consequence of rhinitis, may help save resources for managing rhinitis. This type of disorder affects up to 15% of the general population.

In the first experimental step, the phlegm secretion must be accurately measured. That is, it is necessary to show what is “blown out” of the nose in patients with rhinitis (FIG. 8). The treatment goal is to reduce the accumulation of dense, slimy, viscous phlegm, which will otherwise ooze out of the nasal cavity. In practice, it was discovered that if treatment can reduce individual blowouts to <0.25 g/event, the subject with rhinitis becomes essentially “disease-free” and is able to have normal daily activities and sleep well at night. Blowouts with a weight of >0.35 g/event indicate that the nasal cavity is threatened with the deposition of phlegm and subsequent blockage. The top-loading microbalance method for quantifying phlegm weight is an excellent monitor of rhinitis intensity.

FIG. 8. Measurement of secretions and exudates from the nasal cavity. Subjects were instructed to blow into a pre-weighed paper tissue and record the change in weight of the tissue after blowing. The tissue was inspected and marked as clear, with streaks of exudates, or dense gel. Surprisingly, the nasal blowouts fell into three distinct categories of weights of 0.06 g, 0.20 g and 0.47 g that were easy to recognize (FIG. 8). The weight of the blowouts thus provided an objective index of nasal cavity inflammation.

Case Study 3 and 4 describe breakthrough experiments. A lower dose was formulated and rinsing for 5-sec became the standard procedure. A subject with severe rhinitis, confirmed by a CT scan and endoscopic exam by an ENT physician, volunteered and conscientiously recorded phlegm weights. The results in FIG. 8 clearly show that the preferred embodiments inhibit the daily secretion of phlegm. These observations relied on the NasoNeb® delivery system and the top-loading balance for quantifying phlegm production.

FIG. 9. Inhibition of nasal secretions in a subject with rhinitis by topical administration of the preferred embodiments D-Ala-OiPr and GlyOiPr with a Nasoneb® apparatus. The test substances were rinsed into the nasal cavity (dissolved 2 mg/ml and 3 mg/mL in 1% ethanol and 2% polysorbate 80) for 5 sec at t=0, 2, and 5 hr. The data points represent secretions recorded for 10-15 hr per day. These points were fitted by linear regression (GraphPad Prism), and the 10-hr secretion measured (avg.±s.e.m) as vehicle 3.53±0:44 g, D-Ala-OiPr 1.700:27 g; GlyOiPr 1.32±0.32 g, with n=6, 5, 5 observations per group, respectively. The nasal secretion-10 hr unit for D-Ala-OiPr and GlyOiPr values were significantly lower (P<0.001) than the vehicle when analyzed by the Kruskal-Wallis test for non-parametric data.

The inhibition of nasal secretions could be measured not only as the amount secreted over 10-hour, but also as the volume per blowout, as shown in FIG. 10. The blowout per individual event is important because it is the definite opinion of the participants that if the blowout exceeded ˜0.35 g per event, then the phlegm is visible, unpleasant, disturbing and more likely to be easily dried in the nose and form obstructive snot.

FIG. 10. Weights of individual blowouts of nasal secretions in a subject with rhinitis after topical administration of D-Ala-OiPr and GlyOiPr. (with a Nasoneb apparatus. The vehicle or test substances were rinsed into the nasal cavity (0 mg/mL, 2 mg/mL and 3 mg/mL, respectively, for 5 sec at t=0, 2, and 5 hr). The data points are for individual “blowouts” from the nose, measured with tissue paper and balance. The data are plotted on a semi-log scale, so zero values are not shown. The average #s.e.m. for blowouts were: vehicle 0.40±0.03 g, D-Ala-OiPr 0.19±0.02 g, GlyOiPr 0.19±0.02 g, with n=70, 44, 42 observations per group, respectively. The blowout unit for D-Ala-OiPr and GlyOiPr were significantly lower (P<0.0001) than the vehicle when analyzed by the Kruskal-Wallis test for non-parametric data.

### Significance of Stopping Mucus Hyersecretion

Allergic rhinitis, vasomotor rhinitis, and chronic rhinosinusitis are generally viewed as long-term disorders (>12 weeks), with changes in the immune response to nasal injury. The corticosteroids target the immune cells of inflammation, and the newer monoclonal antibodies for chronic rhinosinusitis, dupilumab, and mepolizumab target the interleukin family of inflammatory mediators. The results herein suggest that rhinitis may be treatable as an acute inflammatory disease of the nasal mucosa, much like a mosquito bite or a pustule. The treatment goal is to reduce phlegm production below 0.25 g per blowout event. The subject's nasal mucosa then begins to recover and resurrects to clean and refreshed breathing. This resurrection can become permanent and maintained. This change may occur within three to five days after treatment. Phlegm is the culprit for chronic pathology. Reducing phlegm production may halt and reverse the course of the disease.

### Case Study 1.

A 58-year-old male venture capitalist located in Beijing, China, suffered from rhinosinusitis for at least five years. He received dietary supplements, intranasal steroids, oral steroids, and antihistamines, but these were ineffective. Based on religious beliefs of natural balance and energy flow, he refused endoscopic surgery or monoclonal antibody drug injections. However, the severity of his facial headaches, uncomfortable breathing, and sleep loss affected his ability to function. In an interview, the individual had dark circles under his eyes, a tired appearance, and a flushed, puffy look, especially about the eyes. He blew his nose constantly and appeared to breathe partially via his mouth. He was short-tempered and miserable.

The subject was given a NasoNeb® nebulizer (unavailable in China) and five individual bottles containing 15 mL of a 1% GlyOiPr solution (suspended in 2% Tween 80, 5% ethanol, and 92% water). He was shown how to load the NasoNeb canister with about 2 mL of liquid and activate the device for a 3-second dose into each nostril. The recommended dosage was three times a day, starting in the morning and at about 6-hour intervals, and for five days.

The subject reported an immediate sensation of comfortable cooling in the nose and the ability to have unimpeded breathing. Over several days of use, the subject noted that nasal discharge, measured by the number of wipes used to blow the nose, decreased by at least 50%. The subject slept well. His colleagues commented on his improved outward appearance, demeanor, and temperament. They said he had not smiled for a long time and was now more pleasant. The subject was advised to continue using the solution three times per day for another week, then taper off the dosage to twice a day, and then use it as needed. After two months of use, the subject reports that he completely controls his nasal problems. He can breathe comfortably and sleep well. He said there continues to be some watery mucus discharge from his nose, but blowing the nose is sufficient for clearance. The headaches are gone, and his breathing is unimpeded. He sleeps well at night.

### Case Study 2.

An 80-year-old male with a 15-year history of Parkinson's disease had to move to an assisted living community to receive more personal care services and medical support. He had his apartment and was quite happy, but after six months, he noticed that his seasonal allergic rhinitis had become perennial and that he was constantly blowing his nose. He counted, on average, about using 20 Kleenex wipes per day. His rhinitis worsened; he could not breathe comfortably at night, woke up with choking sensations, and felt daytime headaches and facial pressure. His mouth felt parched and tasted foul because of his mouth breathing. The nasal discharge became viscous with more mucus. He was diagnosed with chronic rhinosinusitis without nasal polyps and prescribed intranasal and oral steroids, but they were ineffective: He deduced that reactions to potent detergents used in his living facility contributed to his increased nasal sensitivity. However, the housing staff could not change the use of these detergents because of the need for sanitation. Upon consultation with his trusted personal physician, it was decided that he had severe vasomotor rhinitis, but endoscopic sinus surgery may not relieve his symptoms or receive medical insurance coverage.

The subject agreed to using the NasoNeb nebulizer with a cooling agent. He was given a NasoNeb nebulizer and three bottles, each containing 15 ml of a 1% GlyOiPr solution (suspended in 2% Tween 80, 5% ethanol, and 92% water). He was shown how to load the NasoNeb canister with about 2 mL of liquid and activate the device for a 3-second dose into each nostril. The recommended dosage was three times a day, starting in the morning and at about 6-hour intervals, and for five days. After a 5-day trial, the subject noted a significant improvement in sleep and comfortable breathing. The number of Kleenex used was now about five per day. His headaches were gone, and his appetite improved. He was advised to continue using the Nebulizer/coolant for another week and then on an as-needed basis. His family has noticed that the subject has become less withdrawn, more engaging, and interested in his surroundings.

### Case Study 3.

A 52-year-old female advertising executive worked in a big city and was responsible for developing logos and sales brochures for consumer products. In the past three years, she assumed duties for dishwater detergents and other household cleaners. She was also assigned to create advertisements for dog products, such as carpet cleaners. The subject had a long history of allergic rhinitis, which she managed with intranasal steroids and an occasional antihistamine. However, she noticed her rhinitis worsened as she sampled and tested products. A particular dishwater detergent aggravated her rhinitis after herbal oils replaced its chemical scent. Exposure to the detergent's aerosol triggered copious secretion of a slimy yellow discharge from her nose. A carpet cleanser and a mold and mildew cleanser caused similar problems. An ENT doctor diagnosed her case as chronic rhinosinusitis without polyps. The doctor's report noted that she had frequent headaches, nasal purulence, nasal congestion, hyposmia, and sleep difficulty. Matters reached a crisis stage when high-resolution photographs of her at a business speech clearly showed white encrustations on her nostrils.

The subject volunteered for a trial with GlyOiPr. She was given a NasoNeb® machine and a plastic squeeze bottle containing 50 mL of GlyOiPr at 3 mg/mL suspended in 1% ethanol/2% polysorbate 80. She learned how to load the canister and activate the pump. Then, she was instructed to rinse her nose thoroughly first thing in the morning and then use the Nebulizer at 5 sec per nostril to deliver the solution. The procedure was to be repeated at 2 hours and 5 hours later to total three daily activations. The subject was interviewed after dosing for three days. She said breathing was much easier at night after the dosing, and she slept well. Nasal secretions were still present but less in volume. She felt more energetic, and the number of Kleenex tissues she used appeared to decline by half. She was encouraged to use the Nebulizer as needed but not to exceed three daily doses. Three weeks after the start of the experiment, she reported that she was in complete control of her rhinitis. She would do a single dose at night before sleep and one in the morning when needed. She no longer felt congested, and the headaches were gone. The slimy phlegm in the nostrils was gone, and crusting was absent. Her nose felt clean. Her social life improved.

### Case Study 4

A 78-year-old male pharmacologist had a 3-year history of moderate to severe non-allergic (vasomotor) rhinitis. His main complaint was episodes at night when he would wake up and not be able to breathe comfortably. Sleep apnea and lung disorders were ruled out, and the diagnosis was rhinosinusitis. This was confirmed by a computerized tomography scan and endoscopic examination by a qualified ENT specialist. He agreed to test the preferred embodiments GlyOnPr, GlyOiPr, D-Ala-OnPr, and D-Ala-OiPr systematically, one after the other. After testing, he concluded that it was too early to decide on the best agent, but all provided relief from the “stuffiness” episodes that he experienced, and the treatments improved his sleep.

He said the mechanism of drug action should be viewed in a historical context. He wrote: “The history of drug discovery depends on a basic understanding of pathophysiology. Thus, Paul Ehrlich, Gerhard Domagk, and Alexander Fleming discovered arsphenamine for syphilis, sulfonamides for bacterial infections, and penicillin; respectively, only after Louis Pasteur and Robert Koch established the germ theory of disease. Synthetic drug antagonists evolved from Bovet's antihistamines, leading to H₂-receptors for acid secretion. Drug receptor antagonists were in vogue, but remember, the world's best drug, morphine, is an agonist, and the cooling agents are agonists. With an increased understanding of molecular biology, we now have monoclonal antibodies to counter cytokines and inhibitors of Janus kinase. However, new drugs for pain and discomfort have yet to catch up. The TRP receptors V1, A1, and M8 appeared-25 years ago. No new drug candidates from TRP are in the pipeline. VapoRub® has existed since 1911 but is not suited for chronic stuffiness. It is time to move forward with new ideas. The controlled nebulizer permits accurate dosing and delivery to the critical receptor sites. Discovery moves forward when the testing methods become quantitative. Separating the components of rhinitis into stuffiness, sniffles, and mucus secretion is a step in the right direction.”

### Case Study 5.

The recognition that inhibition of mucus hypersecretion might be a valuable and unique pharmacological attribute of TRPM8 agonists prompted the applicant to re-examine such compounds in his inventory. CPS-125 was described by Wei in U.S. Pat. No. 7,417,048 (August 2008), and its structure is in Table 10. This molecule incorporates a sulfamoyl group to increase water solubility and is sulfadiazine coupled to p-menthanecarboxamide. In a range-finding study, CPS-125 was suspended in 1% Ethanol/2% Polysorbate 80/saline and titrated using the Up-Down method of Dixon (Annu. Rev. Pharmacol. Toxicol. 20:441-462.1980) with nasal mucus secretion as the endpoint. The effective concentration for reducing secretions by +80% was 8 mg/ml of CPS-125. The related analogs coupled to sulfanilamide, sulfadimethoxine, sulfisoxazole, sulfameter, and sulfamethoxypyridazine were at least three times less active. There is considerable latitude in how heterocyclic rings of different polarity may be selected and coupled to the sulfamoyl group, so further exploration may reveal more active analogs than CPS-195. Such heterocyclic structures include pyrrole, furan, oxane, thiophene, thioxazole, and oxazole derivatives.

Among the 25+ compounds tested so far, CPS-125 had the longest duration of action. CPS-125 was active when administered as drops or in a manually activated spray. The nose is pinched and released after instilling 0.3 to 0.5 mL into the nostrils. After pinch and release, the reflex sniff or snort enhances delivery of the drops to the ostiomeatal complex. Using a power-assisted nebulizer enhances delivery of CPS-125. CPS-125's molecular weight of 416 Daltons is the highest of the tested compounds. Normally, large lipophilic molecules are not expected to penetrate secretions to reach target. Apparently, the sulfamoyl group confers the qualities of penetration, stability, and retention to make it a viable lead candidate for inhibition of mucus hypersecretion.

### Case Study 6.

A 53-year-old male was diagnosed with chronic rhinosinusitis with nasal polyps according to standard criteria and treated with intranasal corticosteroids. Over a three-month treatment, his symptoms of purulent nasal discharge and sense of nasal obstruction diminished, and his CRS was under control. However, the subject complained of a moderate to severe dry mouth upon awakening each morning. This condition was sufficiently severe that he felt he was losing his sense of taste for food. Drinking acid juices in the morning helped alleviate the dry mouth, but the relief was short-lived, and the persistence of the dry mouth was a continual source of aggravation. This subject approached the applicant for a possible solution to a recurrent dry mouth.

Upon further discussion and analysis of the subject's symptoms, it was hypothesized that the subject's nasal polyps may be physically blocking smooth (lamellar) airflow and that a recumbent position at night during sleep may favor breathing via the mouth; hence, the dry mouth syndrome. As hypersecretion of mucus was not the principal event, it was decided that a long-acting p-menthanecarboxamide, WS-12, might work well for the dry mouth. WS-12 was first discovered by the applicant to be a potent TRPM8 receptor agonist (Reynolds and Polakis, U.S. Pat. No. 7,893,072), and he supplied this reagent for testing and publications [see Bödding et al. (Cell Calcium. 42:618-28, 2007), and Sherkheli (J. Pharm. Pharmaceu. Sci. 13:242-53, 2010)]. WS-12 has weak cooling effects on the skin. WS-12 was later approved as a food additive (FEMA) for comestibles such as chewing gum. WS-12 is available online for about $USD 15 per 100 g. WS-12 was prepared at 10 mg/ml in 1% ethanol and 2% polysorbate 80 suspensions for a 5-see nebulization using the NasoNeb® apparatus in a volunteer subject and administered once before sleep.

Surprisingly; this subject's dry mouth symptoms disappeared after three days of use. The subject now uses the WS-12 on an occasional as-needed basis. The nasal polyps are still present upon endoscopic examination. The WS-12 chemical structure is ideal for use in obstructed breathing because it has an aryl moiety that increases lipophilicity (higher octanol-water partition coefficient). Lipophilicity increases retention in the nasal mucosa; thus, its action is long-lasting. WS-12 does not have sharp, penetrating, refreshing coolness of the isopropyl ester analogs. Its actions are more gentle, with a mild coolness on the nasal surfaces. Activity on dry mouth is seen at a dosing concentration of 10 mg/mL, but an electronically powered nebulizer is required for delivery to the ostiomeatal complex. Manual spraying of WS-12 does not work well. Among the p-menthanecarboxamides WS-12 has the highest octanol/water partition coefficient (see Table 8, Wei U.S. Pat. No. 8,258,320), so its access to the TRPM8 receptors may be hindered by its solubility.

This is the first instance wherein a cooling TRPM8 p-menthanecarboxamide agonist was found to have benefits for the dry mouth of obstructed breathing in the absence of overt nasal mucosal inflammation. The WS-12 type of aryl-substituted p-menthanecarboxamides will likely benefit infant and toddler (1 to 3 years) rhinitis because the absence of penetrating refreshing cool is less likely to cause serous (watery) rhinorrhea and obstruct breathing. Dendreon [Natarajan et al., U.S. Pat. No. 8,362,264 (2013)] thoroughly analyzed the structure-activity relationships of p-methane carboxamides containing aryl substitution(s). However, the lead candidate D-2363 selected by Dendreon for clinical trials may require further optimization because it elevated troponin plasma levels in human volunteers. Elevated troponin indicates damage to the myocardium. The compounds in U.S. Pat. No. 8,362,264 are incorporated herein by reference. An alternative to WS-12 for these applications may be FEMA 4549 (Table 10) and FEMA 4684 which have similar structural characteristics as WS-12. FEMA 4549 is used in a skin cooling combination product called Evercool®. Its TRPM8 receptor potency is similar to that of GlyOiPr (Johnson et al. 2018)

### REFERENCES

1. Fokkens W J et al, European position paper on rhinosinusitis and nasal polyps. Rhinology Vol. 58, Supplement 29, February 2020.
2. Hill B. et al., Physiology and pathophysiology of human airway mucus. Physiol. Rev. 102:1757-1836, 2022.
3. Johnson S et al. Trigeminal Receptor Study of High-Intensity Cooling Agents. J Agric Food Chem. 2018; 66 (10): 2319-23.
4. Natarajan et al., Composition and methods for the treatment of Trp-P8 expression. [U.S. Pat. No. 8,362,264 (2013)]
5. Rogers, D. Physiology of Airway Mucus Secretion and Pathophysiology of Hypersecretion: Conference Proceedings. Respiratory Care 52:1134-1149, 2007.
6. Watson et al., New compounds with the menthol cooling effect. J. Soc. Cosmet. Chem. 29:185-200, 1978.
7. Wei, ET. Aryl-substituted derivatives of cycloalkyl and branched chain alkyl-carboxylic acids useful as antinociceptive drugs for peripheral targets U.S. Pat. No. 7,417,048. August 2008.
8. Wei, ET. N-Alkylcarbonyl lactone compounds and their use. U.S. Pat. No. 8,258,320, September 2012.
9. Wei, ET. [((1R,2S,5R)-2-Isopropyl-5-methyl-cyclohexanecarbonyl)-amino]-acetic acid isopropyl ester and related compounds and their use in therapy. U.S. Pat. No. 8,426,463. Apr. 23, 2013.
10. Wei, ET. N-Alkylcarbonyl-amino acid ester compounds and their use for skin irritation, itch and pain. U.S. Pat. No. 8,853,267. October 2014.
11. Wei, ET. Topical agents for the treatment of sensory discomfort in the nasal cavity. U.S. Pat. No. 9,642,868, May 9, 2017.
12. Widdicombe J H, Wine J J. Airway gland structure and function. Physiol Rev. 2015; 95 (4): 1241-319.
13. Wiig H. Pathophysiology of tissue fluid accumulation in inflammation. J Physiol. 2011; 589 (12): 2945-53.
14. Yang, J. M. et al., and ET Wei (2017). A novel TRPM8 agonist relieves dry eye discomfort. 1-15. https://doi.org/10.1186/s12886-017-0495-2

1. A method of treatment of nasal mucus hypersecretion in a subject in need of treatment thereof, comprising:
topically administering a p-menthane carboxamide TRPM8 agonist to the ostiomeatal complex of the subject's nasal cavity, the administration being therapeutically effective to reduce secretion of phlegm.

2. The method as in claim 1 wherein the p-menthane carboxamide TRPM8 agonist is formulated for administration in a liquid suspension of a solid in a liquid or as a solution.

3. The method as in claim 2 wherein the p-menthane carboxamide TRPM8 agonist in liquid suspension is at 0.2 to 2% weight/volume (2 to 20 mg/mL).

4. The method as in claim 1 wherein the delivered volume to the nasal cavity is 0.3 to: 0:8 mL of liquid per nostril per administration.

5. The method as in claim 1 wherein the amount of p-menthane carboxamide TRPM8 agonist delivered into the nostril per dose is 1 to 10 mg.

6. The method as in claim 1 wherein the p-menthane carboxamide TRPM8 agonist is a p-menthanecarboxamide delivered into the nostril with an electronically-controlled nebulizer.

7. The method as in claim 1 wherein the p-menthane carboxamide TRPM8 agonist is selected from the group consisting of (R)-2-[((1R,2S,5R)-2-isopropyl-5-methyl-cyclohexanecarbonyl)-amino]-propionic acid n-propyl ester (D-Ala-OnPr), (R)-2-[((1R,2S,5R)-2-isopropyl-5-methyl-cyclohexanecarbonyl)-amino]-propionic acid i-propyl ester (D-Ala-OiPr), [((1R,2S,5R)-2-isopropyl-5-methyl-cyclohexanecarbonyl)-amino]-acetic acid n-propyl ester (GlyOnPr), and [((1R,2S,5R)-2-isopropyl-5-methyl-yclohexanecarbonyl)-amino]-acetic acid isopropyl ester (GlyOiPr).

8. The method as in claim 1 wherein the p-menthane carboxamide specific TRPM8 agonist is selected from (1R,2S,5R)-2-isopropyl-N-(4-methoxyphenyl)-5-methyl-cyclohexanecarboxamide (FEMA 4681, WS-12), (1R,2S,5R)—N-(2-(pyridin-2-yl)ethyl)-3-p-menthanecarboxamide (FEMA 4549), or (2S,5R)—N-(4-(2-amino-2-oxoethyl)phenyl)-5-methyl-2-(propan-2-yl)cyclohexanelcarboxamide (FEMA 4684).

9. The method as in claim 1 wherein the p-menthane carboxamide TRPM8 agonist is (1R,2S,5R)-2-Isopropyl-5-methyl-cyclohexanecarboxylic acid [4-(pyrimidin-2-ylsulfamoyl)-phenyl]-amide (CPS-125).

10. The method as in claim 1 wherein the treatment of nasal mucus hypersecretion is the treatment of the mucus hypersecretion of viral rhinosinusitis.

11. The method as in claim 1 wherein the treatment of nasal mucus hypersecretion is the treatment of the mucus hypersecretion of acute virat rhinosinusitis.

12. The method as in claim 1 wherein the treatment of nasal mucus hypersecretion is the treatment of the mucus hypersecretion of the common cold.

13. The method as in claim 1 wherein the treatment of nasal mucus hypersecretion is the treatment of the mucus hypersecretion of chronic rhinosinusitis.

14. The method as in claim 1 wherein the treatment of nasal mucus hypersecretion is the treatment of the mucus hypersecretion of allergic and non-allergic rhinitis.
